(19) United States
(12) Patent Application Publication
(10) Pub. No.: US 2015/0353449 A1
(43) Pub. Date: Dec. 10, 2015

(54) PROCESS FOR THE SELECTIVE HYDROGENATION OF ACETYLENE TO ETHYLENE

(71) Applicant: UOP LLC, Des Plaines, IL (US)

(72) Inventors: Timur Voskoboynikov, Arlington Heights, IL (US); Vincent Mezera, Brookfield, IL (US); Paul T. Barger, Arlington Heights, IL (US); Laura E. Leonard, Western Springs, IL (US)

(21) Appl. No.: 14/298,227

(22) Filed: Jun. 6, 2014

(51) Int. Cl. C07C 5/09

(52) U.S. Cl. CPC C07C 5/09

(57) ABSTRACT

A process for a liquid phase selective hydrogenation of acetylene to ethylene in a reaction zone in which acetylene is contacted with hydrogen under hydrogenation conditions and a molar ratio of hydrogen to acetylene in the reaction zone is at least 5, preferably at least 9. A molar ratio of hydrogen to carbon monoxide is preferably approximately 10. The acetylene is preferably absorbed in a solvent.

### FIELD OF THE INVENTION

This invention relates generally to processes for selectively converting alkynes to olefins, and more specifically to processes for the selective hydrogenation of acetylene to ethylene.

### BACKGROUND OF THE INVENTION

Light olefin materials, including ethylene and propylene, represent a large portion of the worldwide demand in the petrochemical industry. Light olefins are used in the production of numerous chemical products, via polymerization, oligomerization, alkylation and other well-known chemical reactions. These light olefins are essential building blocks for the modern petrochemical and chemical industries for the production of items such as polyethylene. Producing large quantities of light olefin material in an economical manner, therefore, is a focus in the petrochemical industry.

The production of light olefins, and in particular ethylene, can be through steam or catalytic cracking processes. The cracking processes take larger hydrocarbons, such as paraffins, and convert the larger hydrocarbons to smaller hydrocarbons products. The primary product is ethylene. However, there are numerous other chemicals produced in the process. Among the many byproducts are hydrogen, methane, acetylene, and ethane.

Historically, naphtha cracking has provided the largest source of ethylene, followed by ethane and propane pyrolysis, cracking, or dehydrogenation. Due to the large demand for ethylene and other light olefinic materials, however, the cost of these traditional feeds has steadily increased.

Energy consumption is another cost factor impacting the pyrolytic production of chemical products from various feedstocks. Over the past several decades, there have been significant improvements in the efficiency of the pyrolysis process that have reduced the costs of production.

More recent attempts to decrease light olefin production costs include utilizing alternative processes and/or feed streams. In one approach, hydrocarbon oxygenates and more specifically methanol or dimethylether (DME) are used as an alternative feedstock for producing light olefin products. Oxygenates can be produced from available materials such as coal, natural gas, recycled plastics, various carbon waste streams from industry and various products and by-products from the agricultural industry. Making methanol and other oxygenates from these types of raw materials is well established and typically includes one or more generally known processes such as the manufacture of synthesis gas using a nickel or cobalt catalyst in a steam reforming step followed by a methanol synthesis step at relatively high pressure using a copper-based catalyst.

Once oxygenates are formed, the process includes catalytically converting oxygenates, such as methanol, into the desired light olefin products in an oxygenate to olefin (OTO) process. Techniques for converting oxygenates, such as methanol to light olefins (MTO), are described in U.S. Pat. No. 4,387,263, which discloses a process that utilizes a catalytic conversion zone containing a zeolitic type catalyst. This indirect route of production is often associated with energy and cost penalties, often reducing the advantage gained by using a less expensive feed material.

Another alternative process used to produce ethylene involves using pyrolysis to convert natural gas to ethylene. U.S. Pat. No. 7,183,451 discloses heating natural gas to a temperature at which a fraction is converted to hydrogen and a hydrocarbon product such as acetylene or ethylene. The product stream is then quenched to stop further reaction and subsequently reacted in the presence of a catalyst to form liquids to be transported.

A similar process is disclosed in U.S. Pat. No. 7,208,647 in which natural gas is combusted under suitable conditions to convert the natural gas into primarily ethylene and acetylene. The acetylene in the gaseous product stream is separated from the remaining products and converted to ethylene.

More recent efforts have focused on the use of supersonic reactors for the pyrolysis of natural gas into acetylene. For example U.S. Pat. Pub. No. 2014/0058149 discloses a reactor in which a fuel is combusted and accelerated to a supersonic speed. Natural gas is injected into the reactor downstream of the supersonic combustion gas stream, and the natural gas is converted into acetylene as an intermediary product. The reaction is quenched with a liquid to stop the reaction and the acetylene may be converted to the desired product ethylene in a hydrogenation zone.

Whether an undesired byproduct or one of the desired products, acetylene will irreversibly bond with many downstream catalysts, in particular with polymerization catalysts. Therefore, the production streams which include acetylene must be treated to remove or reduce the amount of acetylene. Additionally, in those processes that produce acetylene as an intermediary product, the majority of the acetylene must be converted to ethylene. One method of converting or reducing the amount of acetylene is selective hydrogenation.

Selective hydrogenation process can be utilized to reduce the acetylene concentration to a sufficiently low level and can be done in either a gas phase or a liquid phase. Since selective hydrogenation is a highly exothermic reaction, the liquid phase is sometimes preferred as it can better control temperature of the reaction. For example, U.S. Pat. No. 8,460,937 discloses a process in which acetylene is absorbed into a solvent and passed into a reactor in which a catalyst and hydrogen are present. Under proper reactive conditions, the acetylene is converted into ethylene. The molar ratio of hydrogen to acetylene in the reactor is low, never exceeding approximately four.

A byproduct of selective hydrogenation is C₄+ hydrocarbons (hydrocarbons with four or more carbon atoms). The C₄+ hydrocarbons are undesirable because they can accumulate on catalysts causing coke and fouling the catalyst. Additionally, the creation of the C₄+ hydrocarbons needlessly consumes the acetylene and can make ethylene separation from the rest of products more complicated.

Therefore, it would be desirable to have a process which reduces the production of the C₄+ hydrocarbons in a selective hydrogenation of acetylene to ethylene.

It would also be desirable for such a process that is not limited by a specific catalyst.

### SUMMARY OF THE INVENTION

It has been discovered that by obtaining a high ratio of hydrogen to acetylene in a selective hydrogenation of acetylene to ethylene, the selectivity to C₄+ hydrocarbons is lowered and the selectivity to ethylene increases. This effect has been observed over several catalysts.

Therefore, a first embodiment of the invention may be characterized as a method for a liquid phase selective hydrogenation of acetylene to ethylene by: contacting acetylene with hydrogen in the presence of a catalyst under hydrogenation reaction conditions; and, maintaining a molar ratio of hydrogen to acetylene to be at least approximately 5.

A second embodiment of the invention may be characterized as a process for a liquid phase selective hydrogenation of acetylene to ethylene by contacting acetylene with hydrogen in the presence of a catalyst under hydrogenation reaction conditions, wherein a molar ratio of hydrogen to acetylene is at least approximately 5, and, wherein a molar ratio of hydrogen to carbon monoxide is between 0.1 to 30.

These and other embodiments relating to the present invention should be apparent to those of ordinary skill in the art from the following detailed description of the invention.

### DETAILED DESCRIPTION OF THE DRAWINGS

In the attached drawings:

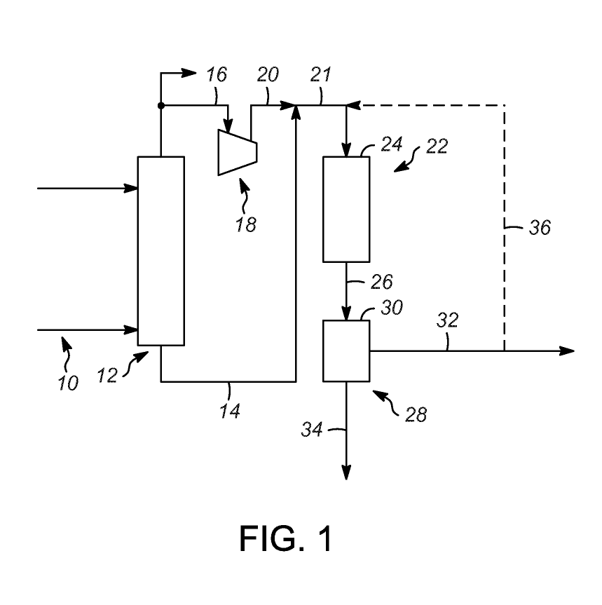

FIG. 1 is a process flow diagram for the liquid phase selective hydrogenation of acetylene to ethylene according to one or more embodiments of the present invention;

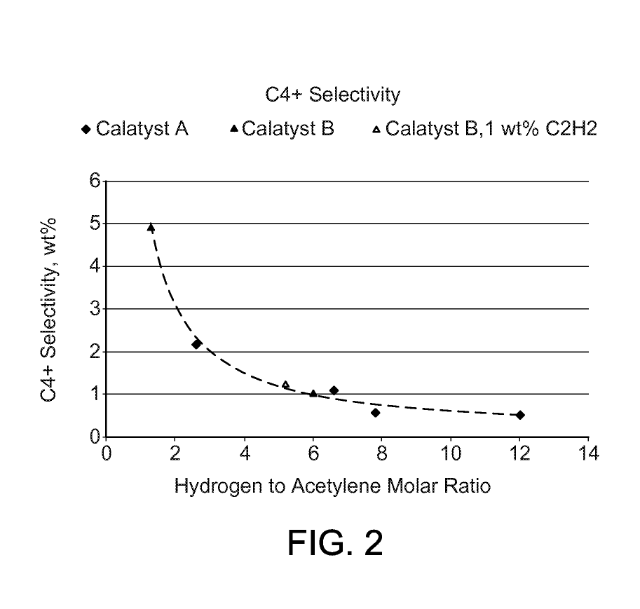

FIG. 2 is a graph showing C₄+ hydrocarbons selectivity based upon one or more embodiments of the present invention;

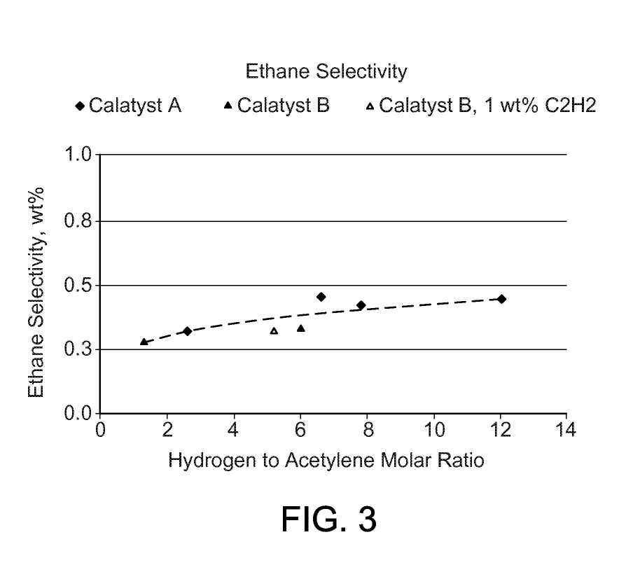

FIG. 3 is a graph showing ethane selectivity based upon one or more embodiments of the present invention;

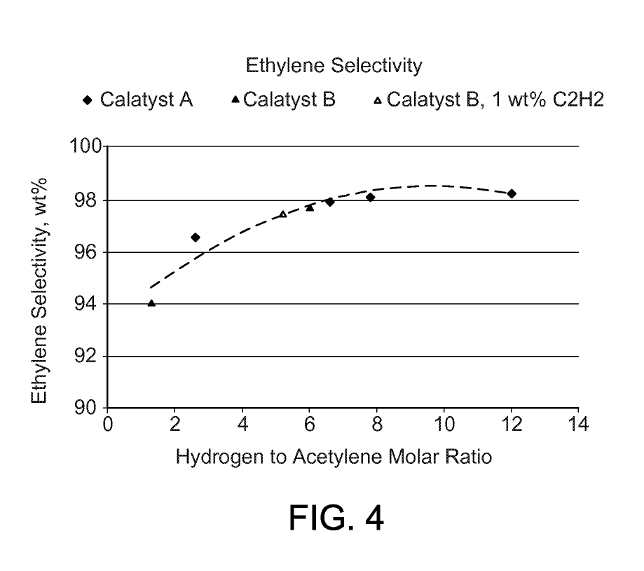

FIG. 4 is a graph showing ethylene selectivity based upon one or more embodiments of the present invention; and,

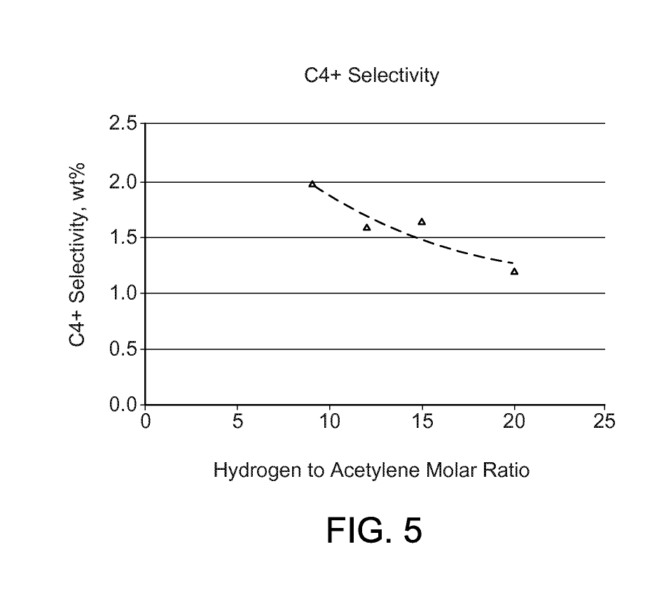

FIG. 5 is another graph showing C₄+ selectivity based upon one or more embodiments of the present invention.

### DETAILED DESCRIPTION OF THE INVENTION

As mentioned above, it has been discovered that for a liquid phase selective hydrogenation of acetylene to ethylene, a high ratio of hydrogen to acetylene, at least 5, preferably at least 7, and most preferably at least 9, will result in a significant decreases of C₄+ hydrocarbon selectivity, without substantial increases in ethane selectivity, and with increases in ethylene selectivity.

An exemplary process for a liquid phase selective hydrogenation of acetylene to ethylene is shown in FIG. 1 in which an acetylene rich vapor steam 10 may be passed to an absorption zone 12. The acetylene in the stream 10 may be obtained from any industrial process. For example, the stream 10 may have only a small amount of acetylene which must be treated to remove the acetylene to avoid damaging a downstream polymerization catalyst. In a preferred embodiment, the acetylene rich vapor stream 10 is obtained from a process in which methane is pyrolyzed in a reactor and more preferably a process in which methane is pyrolyzed in a supersonic reactor to produce acetylene as an intermediate product. In such an embodiment, it is desirable to economically and efficiently convert acetylene to ethylene, and acetylene conversion must be relatively complete. A second, or downstream conversion can be utilized to polish and remove the remaining trace amounts of acetylene. Thus, in the processes in which the acetylene is the intermediary product and ethylene is the desired product, it is undesirable to convert acetylene to products other than ethylene.

Returning to FIG. 1, in the absorption zone 12, acetylene in the acetylene rich vapor stream 10 is absorbed into a solvent, such as n-methyl-2-pyrrolidone (NMP), dimethylformamide (DMF), acetonitrile (ACN), and mixtures thereof. A concentration of acetylene in the solvent is preferably between 0.1% to about 5% by weight, or between about 1% to about 3% by weight.

A first stream 14 being a liquid and comprising solvent and acetylene is removed from the absorption zone 12. A second stream 16 being an acetylene lean vapor stream and comprising at least hydrogen gas is also removed from the absorption zone 12. In order to allow downstream reactors to operate at higher pressures, the second stream 16 (or a portion thereof) may be passed to a compression zone 18 to provide a compressed second stream 20.

The compressed second stream 20 and the first stream 14 from the absorption zone 14 may be combined into a combined stream 21 which is passed to a hydrogenation zone 22. Carbon monoxide may also be passed to the hydrogenation zone 22. While the second stream 16 from the absorption zone 12 may include carbon monoxide, carbon monoxide can also be recovered from a downstream reaction effluent stream or carbon monoxide may be added to the process from another source. The hydrogen and other gases supplied to hydrogenation zone 22 via line 16 may be supplemented by any suitable source of for example purified hydrogen or carbon monoxide.

The hydrogenation zone 22 may include at least one hydrogenation reactor 24. Each hydrogenation reactor 24 includes a hydrogenation catalyst, typically a hydrogenation metal in an amount between 0.01 to 5.0 wt % on a support, wherein the hydrogenation metal is preferably selected from a Group VIII metal. Preferably the metal is platinum (Pt), palladium (Pd), nickel (Ni), or a mixture thereof. More preferably, a Group VIII metal is modified by one or more metals, selected from Group IB through IVA, such as zinc (Zn), indium (In), tin (Sn), lead (Pb), copper (Cu), silver (Ag), gold (Au) in an amount between 0.01 and 5 wt %. Preferred supports are aluminum oxides (aluminas), pure or doped with other metal oxides, synthetic or natural (i.e. clays). More preferred supports are Alpha-Aluminas of various shape and size (i.e. spheres, extrudates), with high degree of conversion to Alpha phase.

In the hydrogenation reactor 24, in the presence of the catalyst, under hydrogenation conditions, the hydrogen reacts with the acetylene to produce ethylene. The hydrogen may be in the second stream 16 from the absorption zone 12, or hydrogen may come from a portion of a downstream reaction effluent, or hydrogen may be added to the process.

Typical hydrogenation reaction conditions in the hydrogenation reactor 24 include a temperature that may range between 50° C. and 250° C., preferably between 100° C. to 200° C. Additionally, the hydrogenation reactor 24 is operated at a high pressure which may range between approximately 0.69 MPa (100 psig) and 3.4 MPa (500 psig), preferably between approximately 1.0 MPa (150 psig) and 2.8 MPa (400 psig). The liquid hour space velocity (LHSV) at the reactor inlet of the hydrogenation reaction can range between 1 and 100 h⁻¹, with preferred ranges being between 5 and 50 h⁻¹, between 5 and 25 h⁻¹, and between 5 and 15 h⁻¹.

The products of this reaction can be recovered from the hydrogenation reactor 24 via a stream 26. The reactor effluent stream 26 is passed to a separation zone 28 which contains, for example, a separator vessel 30.

In the separator vessel 30 of the separation zone 28, the reaction effluents are separated into an overhead vapor stream 32 and a bottoms liquid stream 34. The overhead vapor stream 32 is rich in ethylene and may contain other gases. The further processing of these streams 32, 34 are not necessary for an understanding and practicing of the present invention. However, since the overhead vapor stream 32 may include carbon monoxide and hydrogen, a portion 36 of this stream 32 may be recycled back to the stream 21 entering the hydrogenation zone 22 to provide carbon monoxide and hydrogen for the hydrogenation reactions.

As discussed above, undesirable byproducts of this reaction include C₄+ hydrocarbons. Therefore, in order to minimize the production of the undesirable C₄+ hydrocarbons, the present invention provides a process in which the molar ratio of hydrogen to acetylene in the hydrogenation reactor 24 is at least 5, or at least 6, and preferably at least 7, and most preferably at least 9. Additionally, the molar ratio of carbon monoxide to acetylene in the reaction zone is between about 0.1 to about 30, or between about 0.5 to about 20, or between about 0.5 to about 4. Further, the molar ratio of carbon monoxide to hydrogen may be approximately 10, or may range from 0.1 to 20.

Without being bound to any theory, it is believed that suppression of oligomerization might be related to a reaction rate of hydrogenation. More specifically, hydrogen dissociative adsorption is believed to be a limiting reaction step. By providing more hydrogen it is thought that the rate of hydrogenation is accelerated to the point that no acetylene is left for oligomerization, or the concentration of adjacent adsorbed acetylene species is decreased, thus decreasing the probability of bimolecular oligomerization reactions.

Unexpectedly, it was discovered that such high hydrogen to acetylene ratio leads not only to significant decreases of C₄+ hydrocarbon selectivity, but also does so without substantial increases in ethane selectivity which results in net increase in ethylene selectivity. In order to illustrate the principles of the present invention, a series of experiments are described below. The experimental results are shown in TABLE 1 and FIG. 2, FIG. 3, and FIG. 4. The data surprisingly demonstrates the increased selectivity of the target ethylene product in a selective hydrogenation reaction with a high hydrogen to acetylene ratio, which will be appreciated by those of ordinary skill in the art.

One exemplary catalyst, Catalyst A, was prepared with 0.08 wt % Pd and 0.16% Ag on an alpha alumina support. Catalyst A was tested at 2.5 hr⁻¹ liquid hourly space velocity with a feedstock consisting of 2 wt % acetylene in solvent at 1.72 MPa (250 psig) with a carbon monoxide to acetylene molar ratio of between about 1 to about 4. Acetylene conversion at these conditions was greater than 99%. A second exemplary catalyst, Catalyst B, was prepared was prepared with 0.12 wt % Pd and 0.24% Ag on an alpha alumina support. Catalyst B was tested at 10 hr⁻¹ liquid hourly space velocity with feedstocks consisting of 2 wt % acetylene or 1 wt % acetylene in solvent at 1.72 MPa (250 psig) with a carbon monoxide to acetylene molar ratio of about 0.5 to about 2.5. Acetylene conversion at these conditions was between 96-99%.

The ratio of hydrogen to acetylene, and carbon monoxide to acetylene, the acetylene conversion, as well as the selectivity to some of the products are shown below in TABLE 1 (the selectivity to oxygenates (i.e. acetone, acetaldehyde, etc), does not exceed 1% and is not shown). The C₄+ hydrocarbons selectivity is plotted in FIG. 2 versus the experimental hydrogen to hydrocarbon ratio. As can be seen in TABLE 1, for both catalysts A and B, data was collected at 2 wt % acetylene concentration in the solvent, while additional data for catalyst B was collected at 1 wt % acetylene concentration in the solvent. The ethane selectivity is plotted in FIG. 3 versus the experimental hydrogen to hydrocarbon ratio. The ethylene selectivity is plotted in FIG. 4 versus the experimental hydrogen to hydrocarbon ratio.

TABLE 1

| | Feed | | | | | Sel, wt % | | |
|---|---|---|---|---|---|---|---|---|
| Cat- | wt % | LHSV | Molar ratio | | Conv. | | | Σ |
| alyst | C₂H₂ | hr⁻¹ | H₂:C₂H₂ | CO:C₂H₂ | wt % | C₂ = | C₂ | of C₄+ |
| Cat- | 2 | 2.5 | 2.6 | 1.3 | 100.0 | 96.6 | 0.3 | 2.2 |
| alyst | 2 | 2.5 | 6.6 | 1.3 | 100.0 | 97.9 | 0.5 | 1.1 |
| A | 2 | 2.5 | 7.8 | 3.8 | 99.6 | 98.1 | 0.4 | 0.5 |
| | 2 | 2.5 | 12 | 3.9 | 99.6 | 98.2 | 0.4 | 0.4 |
| Cat- | 2 | 10 | 1.3 | 0.6 | 96.4 | 94.1 | 0.3 | 4.9 |
| alyst | 2 | 10 | 6 | 2.0 | 96.5 | 97.7 | 0.3 | 1.0 |
| B | 1 | 10 | 5.2 | 2.5 | 98.4 | 97.5 | 0.3 | 1.2 |

As will be appreciated and is illustrated in FIG. 2, a significant decrease in C₄+ hydrocarbon selectivity is observed for a variety of catalysts, and conditions. Surprisingly, as shown in FIG. 3, there is no substantial increase in ethane selectivity with the increase in hydrogen to acetylene ratio. As shown in FIG. 4, the selectivity to ethylene at high conversion is substantially improved with increased hydrogen to acetylene ratio.

Therefore it is believed that a significant improvement of selectivity to the desired product may be obtained by operating or maintaining the hydrogenation reactors with a hydrogen to acetylene ratios greater than about 5:1 on a molar basis, or greater than about 6:1, or greater than about 7:1, or greater than about 9:1. This improvement is obtained while maintaining acetylene conversion of greater than about 90%, preferably greater than about 95%, and more preferably greater than about 97%.

Additional experimental data was obtained using a third exemplary catalyst, Catalyst C, with similar properties to Catalyst B and Catalyst C described above albeit with different metal loadings. The exemplary experimental data was obtained at 1.72 MPa (250 psig), 10 LHSV (h⁻¹), 2 wt % acetylene in solvent, varied H₂ to acetylene molar ratio, and 0.9 carbon monoxide to acetylene molar ratio. The results of the additional examples are shown below in TABLE 2 and FIG. 5.

TABLE 2

| | | | Acetylene | Selectivity (wt %) | | |
|---|---|---|---|---|---|---|
| Cat- | Molar ratios | | Conversion | Ethyl- | | Σ |
| alyst | H₂:C₂H₂ | CO:C₂H₂ | wt % | ene | Ethane | of C4+ |
| Cat- | 9 | 0.9 | 98.8 | 97.4 | 0.3 | 2.0 |
| alyst | 12 | 0.9 | 98.6 | 97.0 | 0.3 | 1.6 |
| C | 15 | 0.9 | 98.3 | 97.3 | 0.3 | 1.6 |
| | 20 | 0.9 | 98.2 | 97.8 | 0.3 | 1.2 |

TABLE 2 and FIG. 5 show that even as the hydrogen to acetylene molar ratio is increased as high as 20:1, the C₄+ hydrocarbons selectivity decreases with increasing hydrogen to acetylene ratio without the expected increase in ethane selectivity or decrease in ethylene selectivity.

Therefore, one or more embodiments of the present invention provide a process which decreases C₄₊ hydrocarbons selectivity in a selective hydrogenation of acetylene to ethylene. This will allow for better recovery of the desired products, better use of the acetylene, and less production of undesirable components. One skilled in the art will appreciate that an added benefit of reducing C₄+ hydrocarbons production in the selective hydrogenation of acetylene to ethylene is that lowering C₄+ hydrocarbons byproducts, particularly lowering heavy hydrocarbons which are thought to result in higher coke can lead to longer catalyst life.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understating the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for a liquid phase selective hydrogenation of acetylene to ethylene comprising:
contacting acetylene with hydrogen in a reaction zone in the presence of a catalyst under hydrogenation reaction conditions; and,
maintaining a molar ratio of hydrogen to acetylene in the reaction zone to be at least approximately 5.

2. The process of claim 1 further comprising:
absorbing acetylene in a solvent; and,
passing the mixture of acetylene to the reaction zone.

3. The process of claim 2 wherein the solvent is selected from the group consisting of: n-methyl-2-pyrrolidone;
dimethylformamide; acetonitrile; and, mixtures thereof.

4. The process of claim 3 wherein a concentration of acetylene in the solvent is between 0.1% to about 5% by weight.

5. The process of claim 4 wherein the molar ratio of hydrogen to acetylene in the reaction zone is maintained to be at least 7.

6. The process of claim 1 wherein the molar ratio of hydrogen to acetylene in the reaction zone is maintained to be at least 7.

7. The process of claim 6 wherein a molar ratio of carbon monoxide to acetylene in the reaction zone is between about 0.1 to about 30.

8. The process of claim 1 wherein a molar ratio of carbon monoxide to acetylene in the reaction zone is between about 0.1 to about 30.

9. The process of claim 8 wherein the molar ratio of carbon monoxide to acetylene in the reaction zone is between about 0.2 to about 20.

10. The process of claim 9 wherein the molar ratio of hydrogen to acetylene in the reaction zone is maintained to be at least 7.

11. A process for a liquid phase selective hydrogenation of acetylene to ethylene comprising:
contacting acetylene with hydrogen in a reaction zone in the presence of a catalyst under hydrogenation reaction conditions,
wherein a molar ratio of hydrogen to acetylene in the reaction zone is at least approximately 5, and,
wherein a molar ratio of acetylene to carbon monoxide in the reaction zone is between 0.1 to 30.

12. The process of claim 11 further comprising:
absorbing acetylene in a solvent; and,
passing the mixture of acetylene to the reaction zone.

13. The process of claim 12 wherein a concentration of acetylene in the solvent is between 0.1% to about 5% by weight.

14. The process of claim 13 wherein the solvent is selected from the group consisting of: n-methyl-2-pyrrolidone;
dimethylformamide; acetonitrile; and, mixtures thereof.

15. The process of claim 12 wherein a concentration of acetylene in the solvent is between about 1% to about 3% by weight.

16. The process of claim 15 wherein the molar ratio of hydrogen to acetylene in the reaction zone is at least approximately 9.

17. The process of claim 11 wherein the molar ratio of hydrogen to acetylene in the reaction zone is at least approximately 7.

18. The process of claim 17 wherein the molar ratio of acetylene to carbon monoxide in the reaction zone is between about 0.5 to about 4.

19. The process of claim 11 wherein the molar ratio of hydrogen to acetylene in the reaction zone is at least approximately 9.

20. The process of claim 19 wherein the molar ratio of acetylene to carbon monoxide in the reaction zone is between about 0.5 to about 4.
